(19) United States
(12) Patent Application Publication
(10) Pub. No.: US 2010/0063357 A1
(43) Pub. Date: Mar. 11, 2010

(54) ENDOSCOPE INSERTION AID, ENDOSCOPE APPARATUS AND ENDOSCOPE APPARATUS INSERTION METHOD

(75) Inventor: Atsushi WATANABE, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 12/551,710

(22) Filed: Sep. 1, 2009

(30) Foreign Application Priority Data
Sep. 8, 2008 (JP) 2008-230022

(51) Int. Cl. A61B 1/00

(52) U.S. Cl. 600/114

(57) ABSTRACT

An endoscope insertion aid of the present invention includes a tube body that is inserted into an entrance of an intestinal tract and held by an intestinal wall, a passage hole through which an insertion portion of an endoscope can pass, the endoscope having an insertion path through which a guide wire can pass and a fixing unit that fixes, with the insertion portion passed through the passage hole, a distal end portion of the guide wire protruding from an opening on a distal end side of the insertion portion to the tube body.

### CROSS REFERENCE TO RELATED APPLICATION

This application claims benefit of Japanese Application No. 2008-230022 filed in Japan on Sep. 8, 2008, the contents of which are incorporated by this reference.

### BACKGROUND OF THE INVENTION

1. Field of the Invention

The present invention relates to an endoscope insertion aid, endoscope apparatus and endoscope apparatus insertion method suitable for smoothly inserting an insertion portion of an endoscope into an intestinal tract such as large intestine or small intestine.

2. Description of the Related Art

Endoscopes are generally provided with an operation portion held by an operator to perform various operations and an insertion portion.

The insertion portion is provided with an elongated flexible tubular portion having flexibility that extends from the operation portion, a bending portion linked to a distal end of the flexible tubular portion that can be bent in a horizontal or vertical direction through operations of the operation portion and a distal end rigid portion linked to a distal end of the bending portion.

By an operator et al. inserting the insertion portion of the endoscope from the anus or mouth or nose into the body cavity, the operator et al. can perform an observation, diagnosis, treatment or the like of a predetermined region.

In inserting such an insertion portion of the endoscope into the intestinal tract, a method is conventionally known whereby the operator et al. apply a force to the insertion portion from the outside of a patient's body and pushes the insertion portion into the intestinal tract.

However, with such a pushing method, when the insertion portion of the endoscope is inserted from the anus or mouth into the large intestine or small intestine, since such an intestinal tract is flexible, has a small inner diameter, is long, intricately serpentine, and moreover the intestinal tract is not firmly fixed to the inside of the body cavity, even if the insertion portion of the endoscope is pushed in, when the pushing force is released, the insertion portion is almost pushed back to the original position by a reactive force of the intestinal wall and will not go forward, resulting in a problem that it is hard to insert the insertion portion. Especially a return caused by the reactive force becomes more conspicuous as the insertion portion of the endoscope reaches the depth of the intestinal tract, and therefore when the insertion portion is inserted into the depth, the insertion becomes particularly difficult resulting in the inspection or the like taking a long time.

Therefore, such an endoscope apparatus is proposed that for example, a guide wire is inserted into the intestinal tract first and the insertion portion of an endoscope or catheter or the like is inserted along the guide wire to facilitate the insertion of the insertion portion of the endoscope into the depth of the intestinal tract without simply pushing in the insertion portion by applying a force from outside the patient's body, and such an endoscope apparatus is disclosed, for example, in Japanese Patent Application Laid-Open Publication No. 2004-181250.

### SUMMARY OF THE INVENTION

In summary, an endoscope insertion aid of the present invention is provided with a tube body that is inserted into an entrance of an intestinal tract and held by an intestinal wall, a passage hole through which an insertion portion of an endoscope can pass, the endoscope having an insertion path through which a guide wire can pass, and a fixing unit that fixes, with the insertion portion passed through the passage hole, a distal end portion of the guide wire protruding from an opening on a distal end side of the insertion portion to the tube body.

Furthermore, an endoscope apparatus is provided with a guide wire inserted into an intestinal tract, an insertion portion of an endoscope having an insertion path through which the guide wire is passed, and an endoscope insertion aid that includes a passage hole through which the insertion portion can pass, is inserted into an entrance of the intestinal tract and held by an intestinal wall, and disposed, when the insertion portion is inserted into the intestinal tract, closer to a proximal end side than a distal end side of the insertion portion, wherein the distal end portion of the guide wire protruding from an opening of the insertion path on the distal end side of the insertion portion can be fixed to the endoscope insertion aid.

Furthermore, the endoscope apparatus is provided with a guide wire inserted into an intestinal tract, an insertion portion of an endoscope having an insertion path through which the guide wire can pass, and a fixing member that fixes a distal end portion of the guide wire which is inserted into the insertion path to protrude from an opening on a distal end side of the insertion portion and which is bent into a quasi-U shape, disposed outside the insertion portion and closer to a proximal end side than the distal end portion of the insertion portion.

Furthermore, an endoscope apparatus insertion method includes: a step of causing a guide wire passed through an insertion path in an insertion portion of an endoscope passed through a passage hole of an endoscope insertion aid to protrude from an opening on a distal end side of the insertion portion, a step of bending a region of a distal end side of the protruding guide wire into a quasi-U shape, a step of fixing the distal end of the bent guide wire to a fixing unit of the endoscope insertion aid, and a step of inserting, in a state in which the region protruding from the opening on the distal end side of the insertion portion of the guide wire is bent into a quasi-U shape and the distal end of the guide wire is fixed to the fixing unit, the quasi-U-shaped region of the guide wire and the insertion portion into an intestinal tract.

The above and other objects, features and advantages of the invention will become more clearly understood from the following description referring to the accompanying drawings.

### BRIEF DESCRIPTION OF THE DRAWINGS

FIG. 1 is a diagram showing an overall configuration of an endoscope apparatus provided with an endoscope insertion aid according to a first embodiment of the present invention;

FIG. 2 is a partial cross-sectional view illustrating a configuration of the endoscope insertion aid, insertion portion and guide wire portion in FIG. 1;

FIG. 3 is a perspective view showing a configuration in the vicinity of the distal end portion of the insertion portion in FIG. 1 together with the guide wire;

FIG. 4 is a perspective view showing a state in which the operation portion of the guide wire unit in FIG. 1 is actually operated;

FIG. 5 is a cross-sectional view for illustrating a configuration of the operation portion of the guide wire unit in FIG. 4;

FIG. 6 is a cross-sectional view for illustrating a configuration of the endoscope insertion aid to which a coil pipe of the guide wire unit in FIG. 5 is fixed;

FIG. 7 is a diagram showing a state in which the guide wire portion is sent out by inserting the insertion portion into the sigmoid colon portion in the intestinal tract while bending the insertion portion with the endoscope insertion aid mounted at the anus;

FIG. 8 is a diagram showing a relationship between a bent portion of the sigmoid colon portion and the guide wire portion when the insertion portion has reached the bent portion from the state in FIG. 7;

FIG. 9 is a diagram showing a relationship between the insertion portion and the guide wire portion in the bent portion when viewed from the IX direction in FIG. 8;

FIG. 10 is a diagram showing a state in which the guide wire portion is further moved forward or backward from the state shown in FIG. 9 and the loop portion of the guide wire portion passes through the bent portion;

FIG. 11 is a diagram showing a state in which when the guide wire portion comes out of the bent portion, the hardness of the guide wire portion is increased and the loop portion thereby passes through the bent portion;

FIG. 12 is a diagram showing a state in which the insertion portion is made to move forward along the guide wire portion from the state shown in FIG. 11 and made to pass through the bent portion of the sigmoid colon portion;

FIG. 13 is a diagram showing a state in which when the guide wire portion is pushed in the bent portion, the insertion portion bends between the rectum and the sigmoid colon portion;

FIG. 14 is a diagram showing a state in which the guide wire portion is drawn back from the state shown in FIG. 13, the bending of the insertion portion is released and the insertion portion is allowed to pass through the bent portion again;

FIG. 15 is a diagram showing an overall configuration of an endoscope apparatus provided with an endoscope insertion aid according to a second embodiment of the present invention;

FIG. 16 is a diagram for illustrating the configuration including the guide wire electric drive section and the insertion portion electric drive section making up the main part of the endoscope apparatus in FIG. 15;

FIG. 17 is a partially exploded cross-sectional view of the insertion portion on the distal end side illustrating a configuration of an insertion portion hardness adjusting mechanism provided in the insertion portion in FIG. 14;

FIG. 18 is a cross-sectional view of the insertion portion on the proximal end side in FIG. 17;

FIG. 19 is a cross-sectional view in a direction perpendicular to the insertion axial direction when hardness of the insertion portion is in a normal state;

FIG. 20 is a cross-sectional view in a direction perpendicular to the insertion axial direction when hardness of the insertion portion is in a high hardness state; and

FIG. 21 illustrates the operation which constitutes a feature of the second embodiment.

### DETAILED DESCRIPTION OF THE PREFERRED EMBODIMENTS

Hereinafter, embodiments of the present invention will be explained with reference to the accompanying drawings.

### First Embodiment

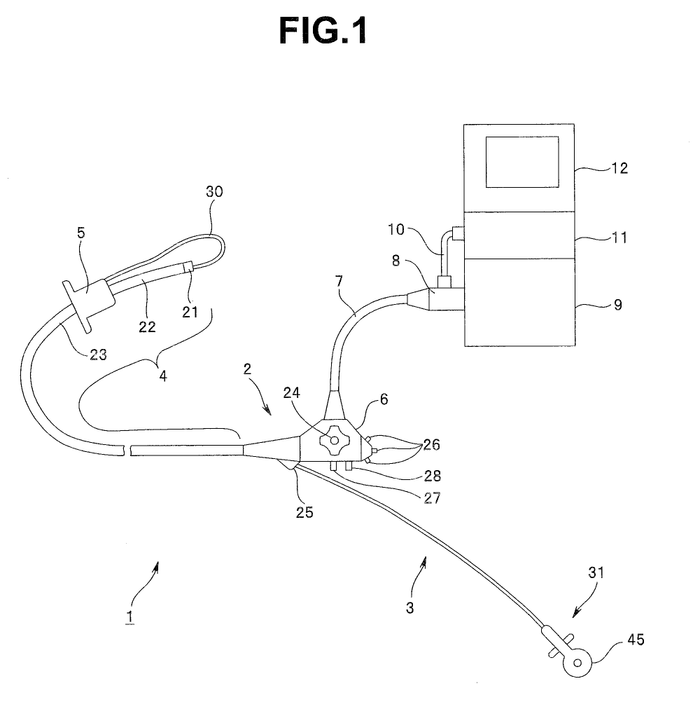

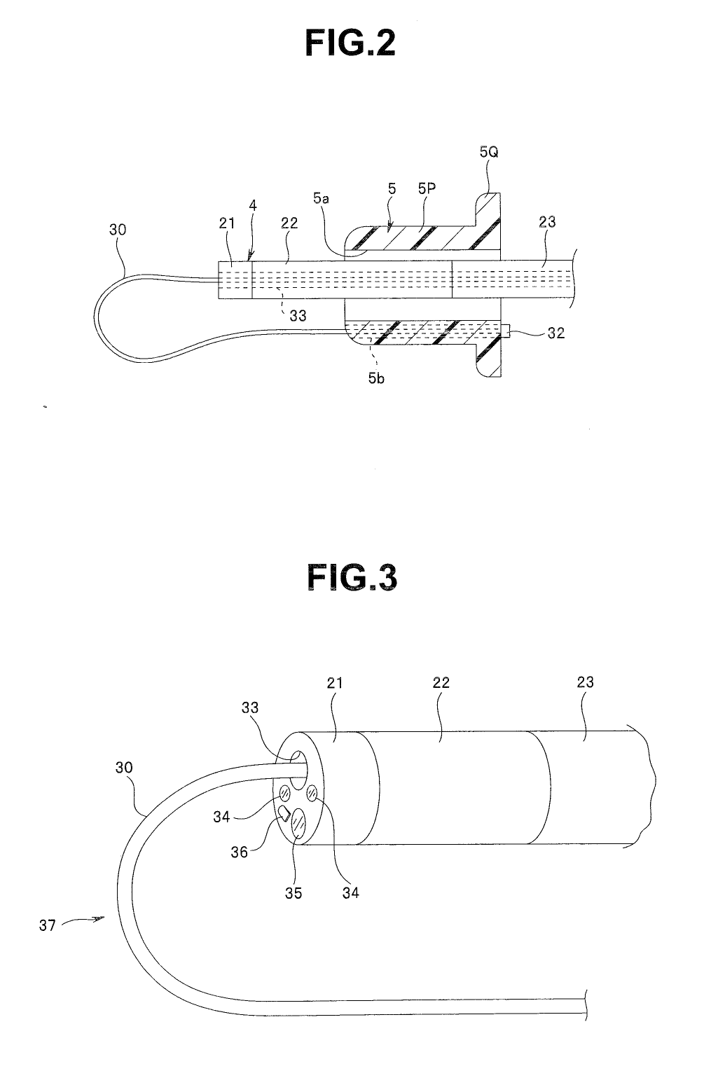

FIG. 1 to FIG. 14 relate to a first embodiment of the present invention, FIG. 1 is a diagram showing an overall configuration of an endoscope apparatus provided with an endoscope insertion aid according to a first embodiment of the present invention, FIG. 2 is a partial cross-sectional view illustrating a configuration of the endoscope insertion aid, insertion portion and guide wire portion in FIG. 1, and FIG. 3 is a perspective view showing a configuration in the vicinity of the insertion portion in FIG. 1 together with the guide wire.

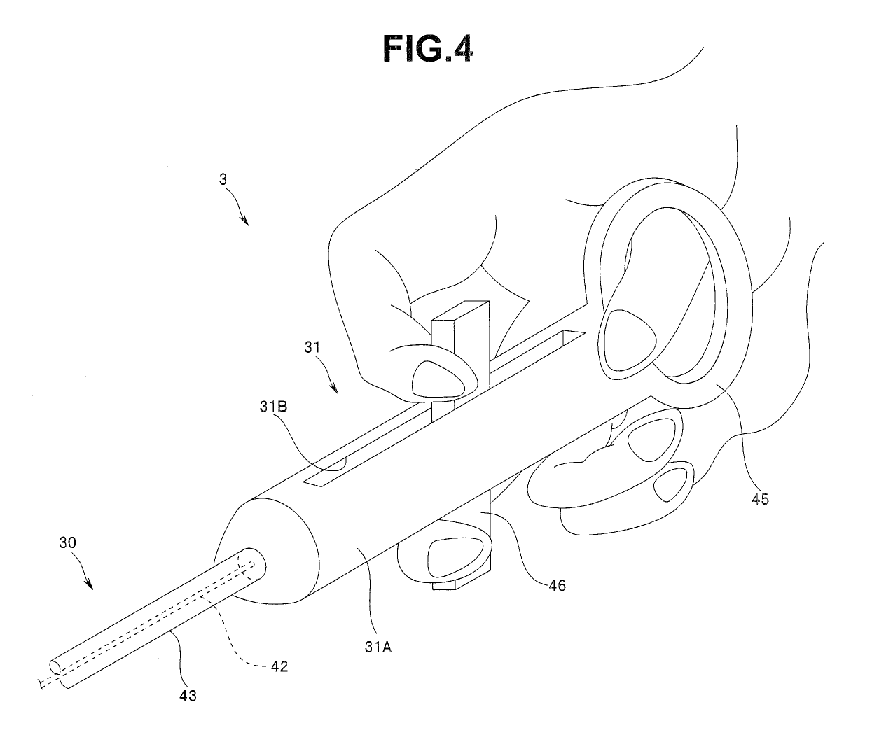

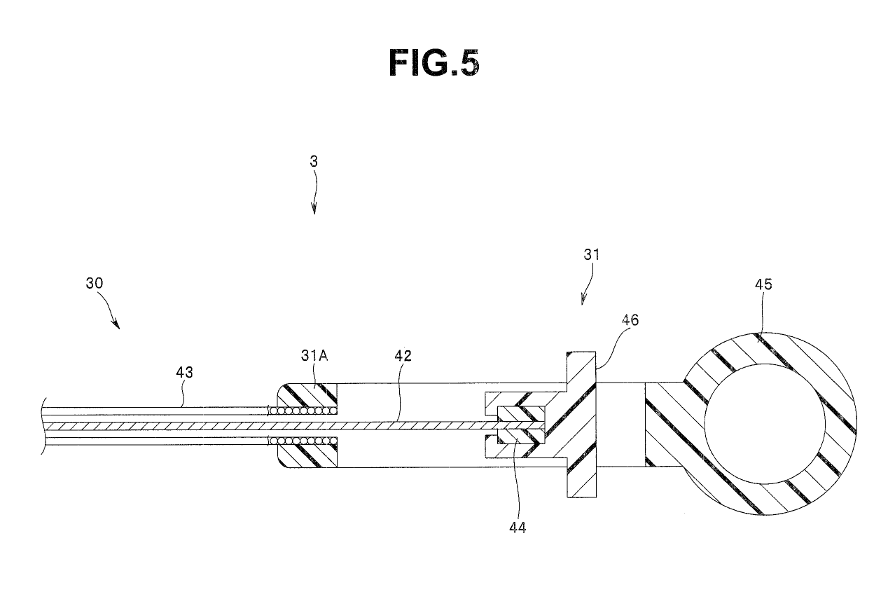

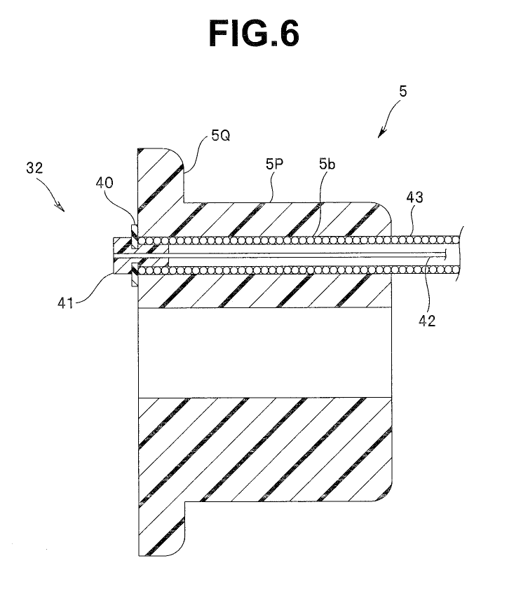

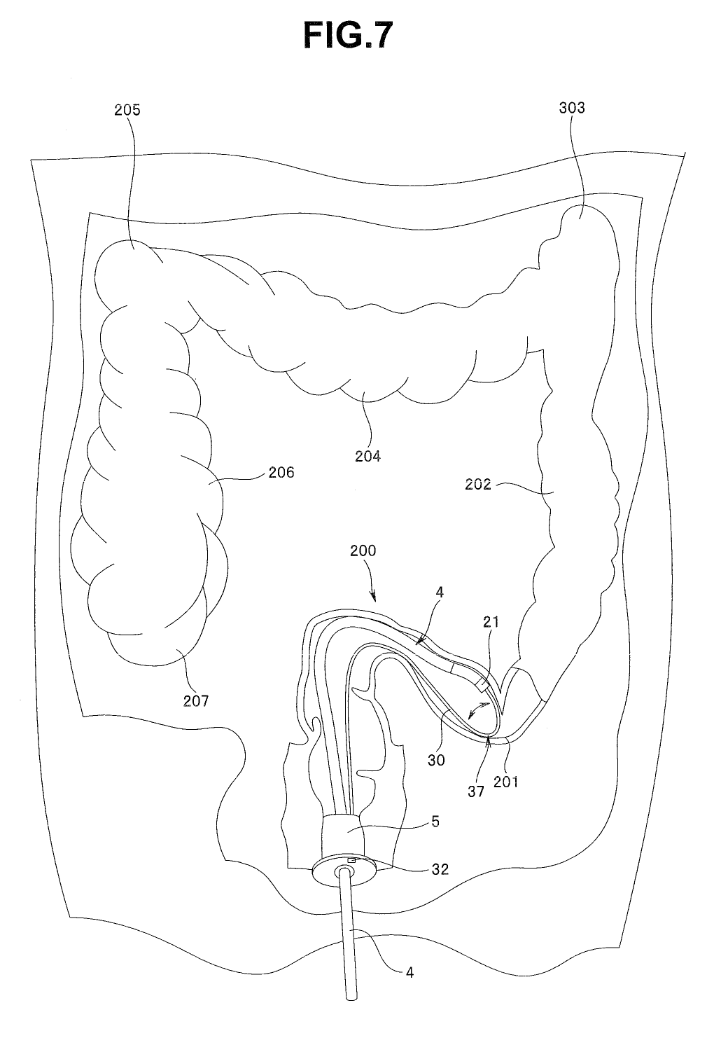

FIG. 4 is a perspective view showing a state in which the operation portion of the guide wire unit in FIG. 1 is actually operated, FIG. 5 is a cross-sectional view illustrating a configuration of the operation portion of the guide wire unit in FIG. 4, FIG. 6 is a cross-sectional view illustrating a configuration of the endoscope insertion aid to which a coil pipe of the guide wire unit in FIG. 5 is fixed. FIG. 7 to FIG. 14 are diagrams for illustrating operations when the insertion portion using the endoscope apparatus of the first embodiment is inserted.

As shown in FIG. 1, the main part of the endoscope apparatus 1 of the first embodiment is configured by including an endoscope 2 inserted in the intestinal tract or the like and used for endoscopy, a guide wire unit 3 having a guide wire portion 30 passed through an insertion path 33 (see FIG. 2) provided in the endoscope 2, an endoscope insertion aid 5 through which the insertion portion 4 of the endoscope 2 can pass and which is a tube body inserted into an entrance in the intestinal tract and held by the intestinal wall, a light source device 9, a video processor 11 and a monitor 12.

The endoscope 2 includes the elongated insertion portion 4 inserted in the intestinal tract or the like, an operation portion 6 provided at a proximal end of the insertion portion 4, and a universal cord 7 that extends from a side of the operation portion 6. A connector 8 is provided at an end of the universal cord 7 and the connector 8 is freely attachable to and detachable from the light source device 9.

The connector 8 is further freely attachable to and detachable from the video processor 11 via a scope cable 10. The video processor 11 performs signal processing on an image pickup signal picked up by the endoscope 2, generates a video signal and outputs the video signal to the monitor 12. In this way, an endoscope image is displayed on the monitor 12.

The insertion portion 4 of the endoscope 2 is configured by including a rigid distal end portion 21, a freely bendable bending portion 22 and a flexible tube 23 having flexibility. The operator can bend the bending portion 22 of the insertion portion 4 in a desired direction by operating the bending operation knob 24 provided in the operation portion 6.

Furthermore, a forceps port 25 of a treatment instrument insertion channel 33 (see FIG. 2) making up an insertion path provided in the insertion portion 4 is provided in the vicinity of the operation portion 6 on the insertion portion 4 side. By inserting a treatment instrument such as forceps through the forceps port 25, it is possible to cause the treatment instrument to protrude from the opening at the distal end of the treatment instrument insertion channel 33 via the treatment instrument insertion channel 33 in the insertion portion 4 and perform biopsy or the like.

Furthermore, the present embodiment has a configuration in which the guide wire portion 30 of the guide wire unit 3 is passed from the forceps port 25 and the guide wire portion 30 can thereby protrude from the opening at the distal end of the treatment instrument insertion channel 33 via the treatment instrument insertion channel 33 (see FIG. 2) in the insertion portion 4. A dedicated insertion path may also be provided in addition to the treatment instrument insertion channel 33 and the guide wire portion 30 may be passed through the insertion path.

Furthermore, a light guide (not shown) is passed through the insertion portion 4 and the light guide is passed up to the connector 8 at an end via the universal cord 7 that extends from the operation portion 6.

Illuminating light is supplied to an end face of the connector 8 from a lamp (not shown) incorporated in the light source device 9. The illuminating light transmitted through the light guide is emitted forward from illumination windows 34 (see FIG. 3) provided at a distal end face of the distal end portion 21 of the insertion portion 4.

Furthermore, as shown in FIG. 1, the operation portion 6 is provided with the bending operation knob 24, the operator can turn the bending operation knob 24 by the fingers of his/her one hand holding the lower end side of the operation portion 6 and can bend the bending portion 22 in an arbitrary direction, upward/downward or rightward/leftward.

The bending portion 22 is made up of a plurality of ring-shaped bending pieces connected in a manner freely rotatable in a longitudinal direction of the bending portion 22. When the bending operation knob 24 is turned, the bending wire tows or loosens the bending pieces making up the bending portion 22 and the bending portion 22 is thereby bent.

Furthermore, as shown in FIG. 1, an air/water supply button 27 and a suction button 28 for performing a suction operation are provided on the plane of the operation portion 6 neighboring the plane on which the bending operation knob 24 is provided.

The operator can supply air or water from the distal end portion 21 of the endoscope 2 by operating the air/water supply button 27. Moreover, the operator can suction a body fluid or other fluid through a channel (not shown) from an opening at a distal end thereof by operating the suction button 28.

Furthermore, a group of a plurality of switches 26 is provided at the top of the operation portion 6. The operator can freeze an endoscope image displayed on the monitor 12 or record an endoscope image displayed on the monitor 12 and so on by operating any one switch of the switch group 26.

Next, the configuration of the endoscope insertion aid 5, insertion portion 4 and guide wire portion 30 in FIG. 1 will be explained using FIG. 2.

As shown in FIG. 2, the endoscope insertion aid 5 is made up of a tube body and configured by including a tube body 5P, a passage hole 5a provided in the tube body 5P through which the insertion portion 4 of the endoscope 2 can pass and a flange 5Q that maintains, when the tube body 5P is attached to the entrance of the intestinal tract, for example, the anus, the attachment state.

The endoscope insertion aid 5 is not limited to the tube body, but may also be made up of, for example, a mesh member wound around the outer periphery of a metallic helical tube and a member like urethane further laminated on the outer periphery thereof or made up using resin such as silicon rubber.

Furthermore, as for the endoscope insertion aid 5, when the insertion portion 4 of the endoscope 2 is inserted into the intestinal tract, the insertion portion 4 is passed through the passage hole 5a, the guide wire portion 30 protruding from the opening of the treatment instrument insertion channel 33 at the distal end portion 21 of the insertion portion 4 is bent into an inverted U shape, and then made to extend in a direction opposite to the insertion direction of the insertion portion 4 and the distal end portion of the guide wire portion 30 is fixed to part of the tube body 5P, for example, the flange 5Q.

The insertion portion 4 passed through the passage hole 5a of the endoscope insertion aid 5 is inserted into the intestinal tract along the guide wire portion 30 protruding from the opening of the treatment instrument insertion channel 33.

The endoscope insertion aid 5 is provided with a fixing unit to fix the guide wire portion 30 of the guide wire unit 3.

The fixing unit is configured by including an insertion channel 5b provided, for example, on part of the outer periphery of the tube body 5P and a guide wire fixing member 32 attached to the flange 5Q of the tube body 5P.

The insertion channel 5b is formed in part of the outer periphery of the tube body 5P along the insertion axial direction of the insertion portion 4 and the distal end portion of the guide wire portion 30 passed through the insertion channel 5b can be fixed to the flange 5Q.

To be more specific, the guide wire portion 30 passed through the insertion channel 5b is fixed by the guide wire fixing member 32 disposed on the outside surface of the flange 5Q, in other words, a surface located outside the anus or the like when the tube body 5P is attached to the anus or the like.

The guide wire fixing member 32 can detachably fix the distal end portion of the guide wire portion 30. The detailed configuration of the guide wire fixing member 32 will be described later.

Next, the configuration of the insertion portion 4 and the guide wire portion 30 passed through the passage hole 5a of the endoscope insertion aid 5 in FIG. 2 will be explained using FIG. 3.

As shown in FIG. 3, the distal end face of the distal end portion 21 of the insertion portion 4 is provided with the opening of the treatment instrument insertion channel 33 and an observation window 35, and two illumination windows 34 and an air/water supply nozzle 36 are provided in the vicinity of the observation window 35.

A light guide that transmits the aforementioned illuminating light via an illumination lens is disposed inside the illumination window 34. Illuminating light generated by the light source device 9 is transmitted through the light guide and emitted out of the illumination window 34 to illuminate the inside of the intestinal tract, which constitutes a range of field of view of the observation window 35.

An objective lens (not shown) is disposed inside the observation window 35 and, for example, a CCD is disposed at the image forming position as an image pickup device. The CCD is designed to photoelectrically convert an optical image in the intestinal tract formed on the image pickup surface of the CCD.

The CCD is connected to a signal cable, the signal cable is passed through the insertion portion 4 or the like and electrically connected to the video processor 11.

Furthermore, as shown in FIG. 3, the guide wire portion 30 is protruded from the opening of the treatment instrument insertion channel 33 disposed at the distal end face of the distal end portion 21, bent into a quasi-U shape in a direction opposite to the insertion direction of the insertion portion 4 and an inverted U-shaped portion 37 is thereby formed.

As describe above, since the distal end portion of the guide wire portion 30 is fixed to the endoscope insertion aid 5, the insertion portion 4 can be moved to the depth in the intestinal tract along the inverted U-shaped portion 37 shown in FIG. 3 by operating the other end side of the guide wire portion 30 passed from the forceps port 25 of the endoscope 2 with an operation portion 31 (see FIG. 4) which will be described later.

Next, the configuration of the guide wire unit 3 having the guide wire portion 30 will be explained using FIG. 4 to FIG. 6.

As shown in FIG. 1 and FIG. 5, the guide wire unit 3 is configured by including the guide wire portion 30 passed through the treatment instrument insertion channel 33 of the insertion portion 4 and the operation portion 31 provided on the proximal end side of the guide wire portion 30.

The guide wire portion 30 is configured by including a wire 42 and a coil pipe 43 that slidably sheathes the wire 42.

The proximal end side of the coil pipe 43 is fixed to the distal end side of an operation portion main body 31A of the operation portion 31 which will be described later (see FIG. 5).

As shown in FIG. 4 and FIG. 5, the operation portion 31 is configured by including the operation portion main body 31A to which the proximal end portion of the guide wire portion 30 is fixed, a towing portion 46 which is engaged with a slide groove 31B of the operation portion main body 31A and is slidably provided in the longitudinal direction of the slide groove 31B and a ring-shaped handle body 45 provided at the rear end of the operation portion main body 31A.

The proximal end portion of the wire 42 passed through the coil pipe 43 is fixed to the distal end side of the towing portion 46 by a stopper 44 by means of brazing or the like. That is, the wire 42 can be relaxed or towed by sliding the towing portion 46 along the slide groove 31B. Relaxing or towing the wire 42 allows the hardness of the guide wire portion 30 to be freely made variable.

Furthermore, pushing in the operation portion 31 itself using the handle body 45 or directly holding and sending the guide wire portion 30 into the forceps port 25 allows the guide wire portion 30 to move in the forward direction of the distal end portion 21. Of course, towing the operation portion 31 itself allows the guide wire portion 30 to be drawn back.

FIG. 4 shows a state in which the operator actually performs operation using the operation portion 31. When he operator actually performs operation using the operation portion 31, for example, the operator inserts his/her thumb into the handle body 45 and hooks his/her forefinger and middle finger at the towing portion 46 that protrudes from the outer periphery of the operation portion main body 31A as shown in FIG. 4.

In this case, to move the guide wire portion 30 forward or backward, the operator only needs to push in the handle body 45 toward the forceps port 25 of the operation portion 6 or draw back the handle body 45 from the forceps port 25. This allows the guide wire portion 30 to move forward or backward through a simple operation.

Furthermore, to make the hardness of the guide wire portion 30 variable, the operator only needs to relax or tow the wire 42 by operating the towing portion 46 forward or toward the operator along the slide groove 31B.

This allows the hardness of the guide wire portion 30 to be made variable through a simple operation. To be more specific, the operator can increase the hardness of the guide wire portion 30 by towing the wire 42 by operating the towing portion 46 toward the operator along the slide groove 31B. By contraries, the operator can reduce the hardness of the guide wire portion 30 by operating the towing portion 46 in the forward direction along the slide groove 31B.

Next, the specific configuration of the guide wire fixing member 32 that detachably fixes the guide wire portion 30 to the endoscope insertion aid 5 will be explained using FIG. 6.

As shown in FIG. 6, the coil pipe 43 of the guide wire portion 30 is passed through the insertion channel 5b of the tube body 5P of the endoscope insertion aid 5 and the distal end portion of the wire 42 in the coil pipe 43 is fixed to a stopper 41 making up the guide wire fixing unit 32 by means of an adhesive or the like.

After fixing the stopper 41 to the distal end portion of the wire 42, for example, a notched ring-shaped guide wire stopper 40 is engaged with the outer periphery of the stopper 41. The outside diameter of the guide wire stopper 40 is made greater than the diameter of the bore of the insertion channel 5b and the guide wire stopper 40 is engaged with the outside of the tube body 5P and fixed, and the wire 42 and stopper 41 can thereby be detachably fixed to the tube body 5P.

A case has been explained above where the guide wire fixing member 32 is made up of the guide wire stopper 40 and stopper 41 or the like, but any configuration may be adopted without being limited thereto if such a configuration allows the distal end portion of the guide wire portion 30 to be engaged and fixed or allows the fixed state to be canceled.

Furthermore, instead of fixing the guide wire portion 30 that protrudes from the flange 5Q via the insertion channel 5b by the guide wire fixing unit 32, the guide wire portion 30 may be fixed and held using other fixing units.

Furthermore, instead of fixing the guide wire portion 30 to the guide wire fixing member 32 of the endoscope insertion aid 5, for example, the guide wire portion 30 protruding from the opening of the treatment instrument insertion channel 33 may be bent into a quasi-U shape and the distal end portion of the guide wire portion 30 may be fixed to another fixing member (not shown) disposed closer to the operator's hand side than the distal end portion of the insertion portion 4 outside the insertion portion 4. The fixing member in this case may be, for example, one disposed outside the body.

Furthermore, the insertion channel 5b through which the guide wire portion 30 is passed may be provided and configured as a separate member on the outer peripheral surface instead of the outer peripheral portion of the tube body 5P. Furthermore, the guide wire portion 30 may be passed through the passage hole 5a of the tube body 5P instead of the insertion channel 5b and may be fixed and held using the guide wire fixing unit 32.

Next, operations when the endoscope 2 according to the present embodiment in such a configuration is inserted into the intestinal tract and endoscopy is performed will be explained using FIG. 7 to FIG. 14.

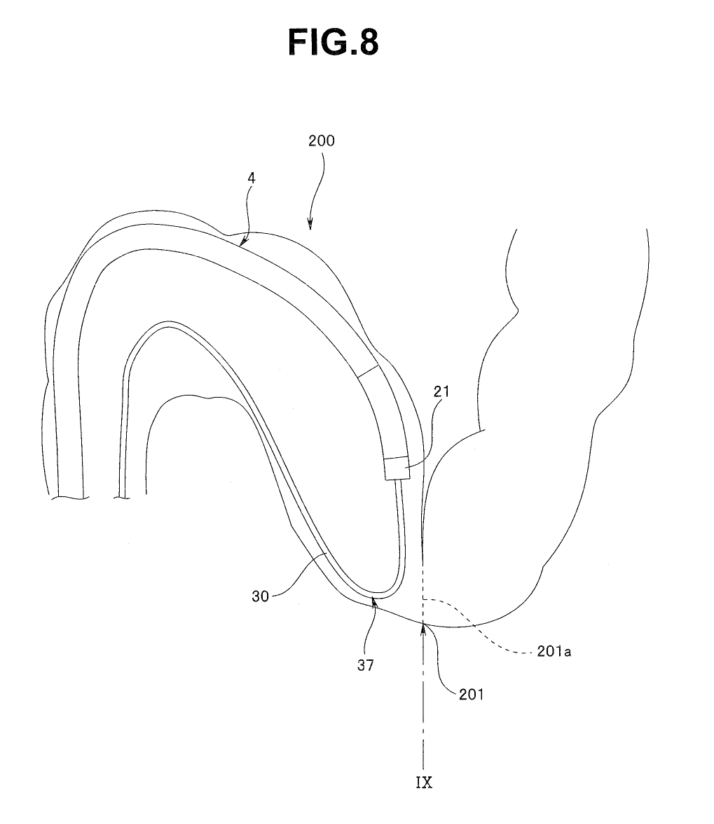

FIG. 7 to FIG. 14 are diagrams for illustrating operations of the insertion portion 4 using the endoscope insertion aid 5 of the first embodiment at the time of insertion, FIG. 7 is a diagram showing a state in which the guide wire portion is sent out by inserting the insertion portion into the sigmoid colon portion in the intestinal tract while bending the insertion portion with the endoscope insertion aid placed at the anus and FIG. 8 is a diagram showing a relationship between a bent portion of the sigmoid colon portion and the guide wire portion when the insertion portion has reached the bent portion from the state in FIG. 7.

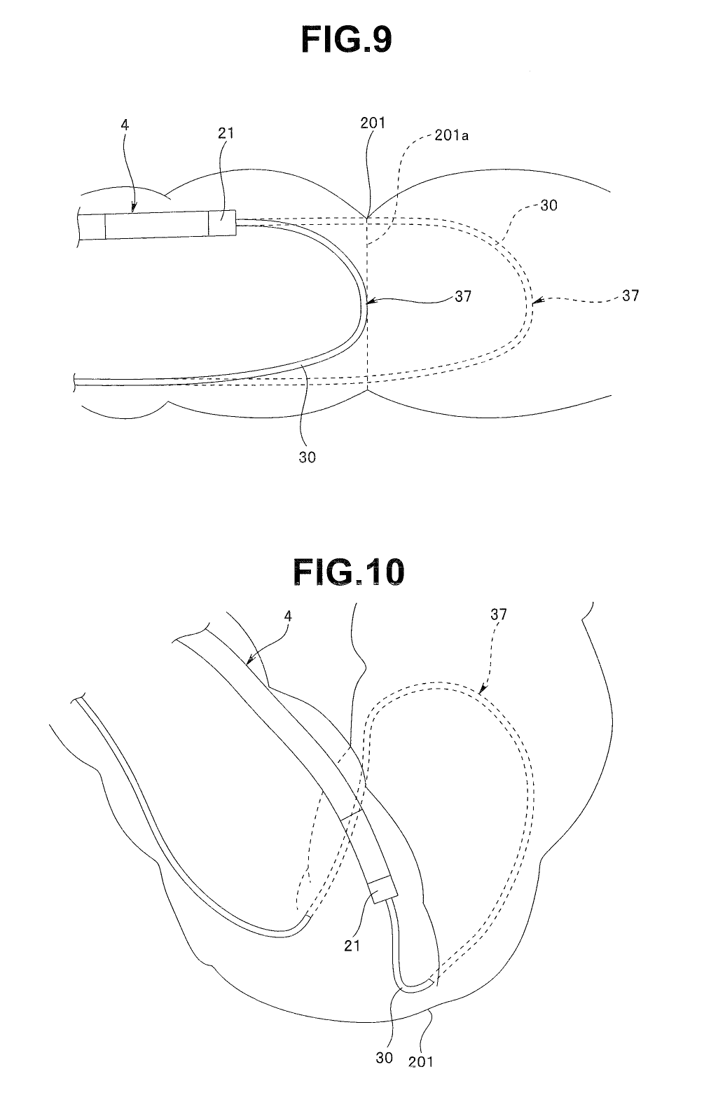

Furthermore, FIG. 9 is a diagram showing a relationship between the insertion portion and the guide wire portion in the bent portion when viewed from the IX direction in FIG. 8 and FIG. 10 is a diagram showing a state in which the guide wire portion is further moved forward or backward from the state shown in FIG. 9 and the loop portion of the guide wire portion passes through the bent portion.

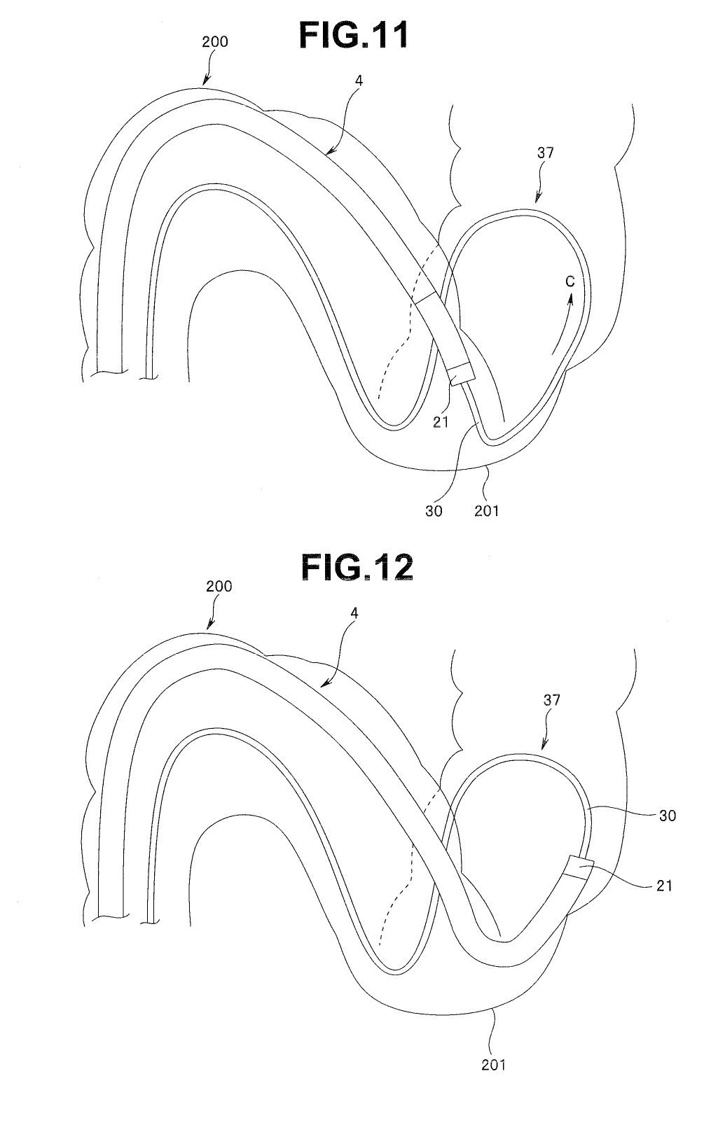

Furthermore, FIG. 11 is a diagram showing a state in which when the guide wire portion comes out of the bent portion, the hardness of the guide wire portion is increased and the loop portion thereby passes through the bent portion and FIG. 12 is a diagram showing a state in which the insertion portion is made to move forward along the guide wire portion from the state shown in FIG. 11 and made to pass through the bent portion of the sigmoid colon portion.

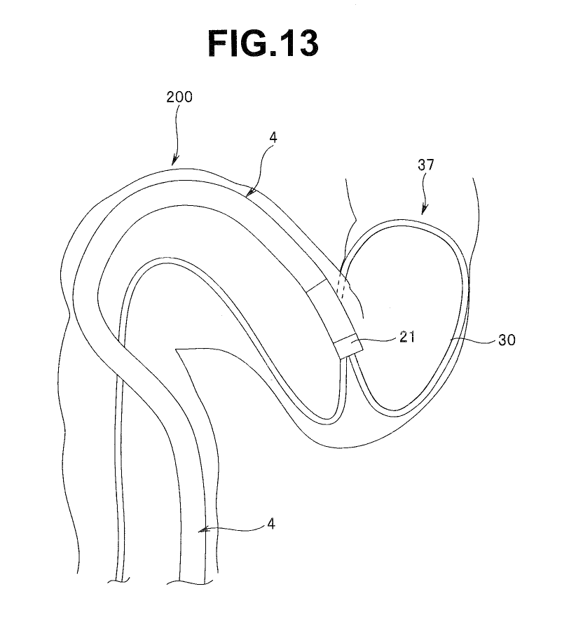

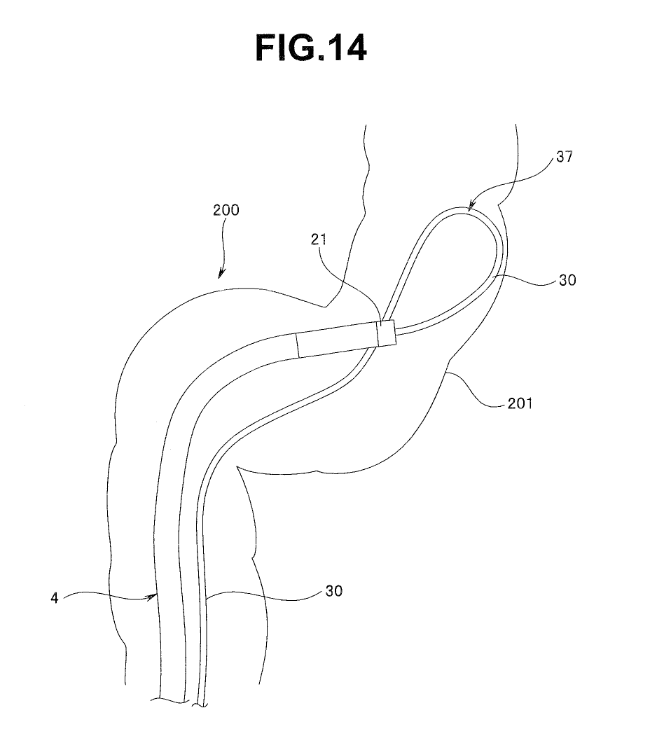

Furthermore, FIG. 13 and FIG. 14 illustrate another insertion method, FIG. 13 is a diagram showing a state in which when the guide wire portion is pushed in the bent portion, the insertion portion bends between the rectum and the sigmoid colon portion and FIG. 14 is a diagram showing a state in which the guide wire portion is drawn back from the state shown in FIG. 13, the bending of the insertion portion is released and the insertion portion is allowed to pass through the bent portion again.

Prior to an endoscopy inspection, the guide wire portion 30 of the guide wire unit 3 is passed through the treatment instrument insertion channel 33 in the insertion portion 4 from the forceps port 25 as shown in FIG. 1.

The insertion portion 4 is then passed through the passage hole 5a of the endoscope insertion aid 5, the guide wire portion 30 protruding from the opening of the distal end portion 21 of the insertion portion 4 is bent into an inverted U shape, then made to extend in a direction opposite to the insertion direction of the insertion portion 4 and the distal end portion of the guide wire portion 30 is fixed to the endoscope insertion aid 5 using the guide wire fixing member 32.

When inspecting the interior of the intestinal tract, the operator inserts and fits the endoscope insertion aid 5 into the anus as shown in FIG. 7 and inserts the insertion portion 4 and guide wire portion 30 into the intestinal tract in this condition.

In this case, the operator inserts the insertion portion 4 of the endoscope 2 and guide wire portion 30 into the anus and rectum while guiding them using the endoscope insertion aid 5 and continues the insertion from the rectum to the sigmoid colon portion 200 through the hand operation and bending operation or the like.

The distal end portion 21 of the insertion portion 4 then reaches the vicinity of the sigmoid colon portion 200 where insertion becomes difficult. Since the bent portion between the rectum and sigmoid colon portion 200 is rich in mobility, the insertion portion 4 is pushed into the intestinal tract as shown in FIG. 7 as the pushing proceeds. Furthermore, a mobility-rich bent portion 201 is formed on the boundary between the sigmoid colon portion 200 and the descending colon portion 202 which is poor in mobility.

The distal end portion of the insertion portion 4 reaches a point just before the bent portion 201 through the bending operation and pushing operation as shown in FIG. 8.

In this case, the insertion portion 4 and guide wire portion 30 are arranged at the point just before the bent portion 201 in the sigmoid colon portion 200 as shown, for example, in FIG. 8 and FIG. 9.

That is, at the same time as the distal end portion 21 of the insertion portion 4 is disposed just before the bent portion 201 in the intestinal tract of the sigmoid colon portion 200 through the operator's pushing operation of the insertion portion 4, the guide wire portion 30 led out of the distal end portion 21 is disposed at a position opposed to the position at which the insertion portion 4 is disposed in the intestinal tract.

In this case, since the inverted U-shaped portion 37 has a large curvature, the entire guide wire portion 30 is bent and the traveling direction thereof never runs reversely as the apparatus described above in Japanese Patent Application Laid-Open Publication No. 2004-181250 and further extends the intestinal tract.

Furthermore, the inverted U-shaped portion 37 of the guide wire portion 30 is disposed in the center of the intestinal tract in this case, and reaches a position 201a corresponding to a slit-shaped portion of the bent portion 201 at the same time.

Here, the operator performs operations of pushing in the handle body 45 of the operation portion 31 in the condition shown in FIG. 8 and FIG. 9 and causes the guide wire portion 30 to move forward from the distal end portion 21 of the insertion portion 4 to the depth of the bent portion 201.

When the guide wire portion 30 is pushed in from the distal end portion 21 of the insertion portion 4 in the intestinal tract, since there is a large distance between the distal end portion 21 from which the guide wire portion 30 protrudes and the guide wire portion 30 that extends in the direction of the insertion portion 4 in the operator's hand side direction via the inverted U-shaped portion 37, the inverted U-shaped portion 37 formed in the guide wire portion 30 expands in contact with the intestinal wall of the bent portion 201.

That is, the guide wire portion 30 expands with the elastic force thereof to the full width of the intestinal tract at the position 201a corresponding to the slit shape of the mobility-rich bent portion 201 as shown by a dotted line in FIG. 9.

In this case, when the guide wire portion 30 is pushed in, the force is transmitted forward more easily and the guide wire portion 30 will not bend easily in the free space in the intestinal tract closer to the operator's hand side than the bent portion 201. Furthermore, the orientation in the plane direction of the inverted U-shaped portion 37 of the guide wire portion 30 is the orientation that follows the shape of the intestinal tract at the position 201a corresponding to the slit shape.

That is, the inverted U-shaped portion 37 of the guide wire portion 30 is formed into a planar shape and since the vertex of the inverted U-shaped portion 37 forms a smooth curve, it never catches on any part of the intestinal tract. Therefore, the inverted U-shaped portion 37 can readily enter the position 201a corresponding to the slit shape of the bent portion 201. Furthermore, since the inverted U-shaped portion 37 can flexibly bend in the vertical direction of the plane, the inverted U-shaped portion 37 enters the depth of the bent portion 201.

Therefore, the inverted U-shaped portion 37 of the guide wire portion 30 operating in this way passes through the position 201a corresponding to the slit shape of the bent portion 201 (see FIG. 9).

Further pushing in the guide wire portion 30 causes the inverted U-shaped portion 37 of the guide wire portion 30 to completely pass through the bent portion 201 as shown in FIG. 10.

That is, the use of the endoscope insertion aid 5 of the present embodiment prevents the guide wire portion 30 from stretching toward the free space in the intestinal tract closer to the operator's hand side than the bent portion 201 at the bent portion 201 as in the case of the conventional configuration and allows the guide wire portion 30 to easily pass through the bent portion 201.

Here, if the guide wire portion 30 is soft without rigidity, the inverted U-shaped portion 37 of the guide wire portion 30 cannot successfully pass through the bent portion 201 or even if the inverted U-shaped portion 37 passes through the bent portion 201, when the insertion portion 4 is further moved forward along the guide wire portion 30 subsequently, the inverted U-shaped portion 37 of the guide wire portion 30 may deviate from the position of the bent portion 201.

However, since the guide wire portion 30 of the present embodiment is configured such that the hardness of a portion within a predetermined range in the longitudinal direction of the guide wire portion 30 can be arbitrarily changed, the inverted U-shaped portion 37 will never deviate from the position of the bent portion 201.

In this case, as hardness variable means that can make the hardness of the guide wire portion 30 variable, for example, the handle body 45 may be configured to be rotatable with respect to the operation portion main body 31A and the wire 42 may be twisted by rotating the handle body 45 to increase the hardness of the guide wire portion 30. Of course, any configuration other than this configuration may also be used as the hardness variable means.

Therefore, when the guide wire portion 30 is configured so as to increase the hardness thereof, the inverted U-shaped portion 37 is made to easily pass through the bent portion 201 by moving the guide wire portion 30 in the direction of an arrow C shown in FIG. 11 in the same way as the operation shown in FIG. 10.

When the operator pushes in the insertion portion 4, the insertion portion 4 passes through the bent portion 201 along the guide wire portion 30 as shown in FIG. 12 and is inserted into the depth. In this case, since the intestinal tract in the depth of the bent portion 201 has been expanded by the inverted U-shaped portion 37 of the guide wire portion 30, the insertion portion 4 can be smoothly inserted.

Furthermore, when the insertion portion 4 is pushed in in a state in which the inverted U-shaped portion 37 of the guide wire portion 30 has passed through the bent portion 201, if the insertion portion 4 is soft, with low hardness, the insertion portion 4 might be bent in the intestinal tract between the rectum and sigmoid colon portion 200 as shown, for example, in FIG. 13.

In such a case, the operator draws back the handle body 45 in the present embodiment. That is, the operator draws back the guide wire portion 30 toward the operator's hand side.

In this case, the operator does not draw back the insertion portion 4 with respect to the intestinal tract. Therefore, the insertion length of the insertion portion 4 remains unchanged in this case.

Furthermore, as shown in FIG. 13, since the guide wire portion 30 has expanded to the full width in the intestinal tract, the guide wire portion 30 does not slide off the intestinal tract and the relative positional relationship between the inverted U-shaped portion 37 and the intestinal tract does not change either.

The insertion portion 4 and the guide wire portion 30 relatively move and the distal end portion 21 of the insertion portion 4 thereby moves forward in the intestinal tract, and when the guide wire portion 30 is drawn back toward the outside of the body, the bending between the insertion portion 4 and guide wire portion 30 can thereby be canceled as shown in FIG. 14.

In this case, the distal end portion 21 of the insertion portion 4 passes through the bent portion 201 and drawing back the insertion portion 4 and the guide wire portion 30 toward the operator's hand side shortens the length from the inverted U-shaped portion 37 to the anus, producing a state of a quasi-straight line.

In the subsequent operation of insertion into the depth of the intestinal tract, using the above described method allows the inverted U-shaped portion 37 to pass through deeper bent portions in the intestinal tract such as the splenic flexure 203 and the hepatic flexure 205 and allows the insertion portion 4 to be inserted likewise.

The operator then repeatedly performs hand operations such as the operation of moving the guide wire portion 30 forward or backward and pushing in the insertion portion or bending operation or the like, and can thereby insert the insertion portion 4 into the vicinity of the cecum portion 207, which is the depth of the intestinal tract after passing through the descending colon portion 202, transverse colon portion 204 and ascending colon 206.

Therefore, the first embodiment provides the endoscope insertion aid 5 and the guide wire unit 3, and thereby allows the guide wire portion 30 pushed into a mobility-rich bent portion where the intestinal tract is narrow to easily pass through the bent portion without extending toward a free space in the intestinal tract closer to the operator's hand side than the bent portion 201 and allows the insertion portion 4 of the endoscope 2 to be smoothly inserted along the guide wire portion 30 that has passed through the bent portion.

The present embodiment assumes the large intestine to be the tube cavity in which the insertion portion 4 of the endoscope 2 provided with the endoscope insertion aid 5 is inserted, but the tube cavity in which the insertion portion 4 is inserted is not limited to the large intestine and may be such a tube cavity from the oral cavity to the esophagus, stomach and small intestine.

### Second Embodiment

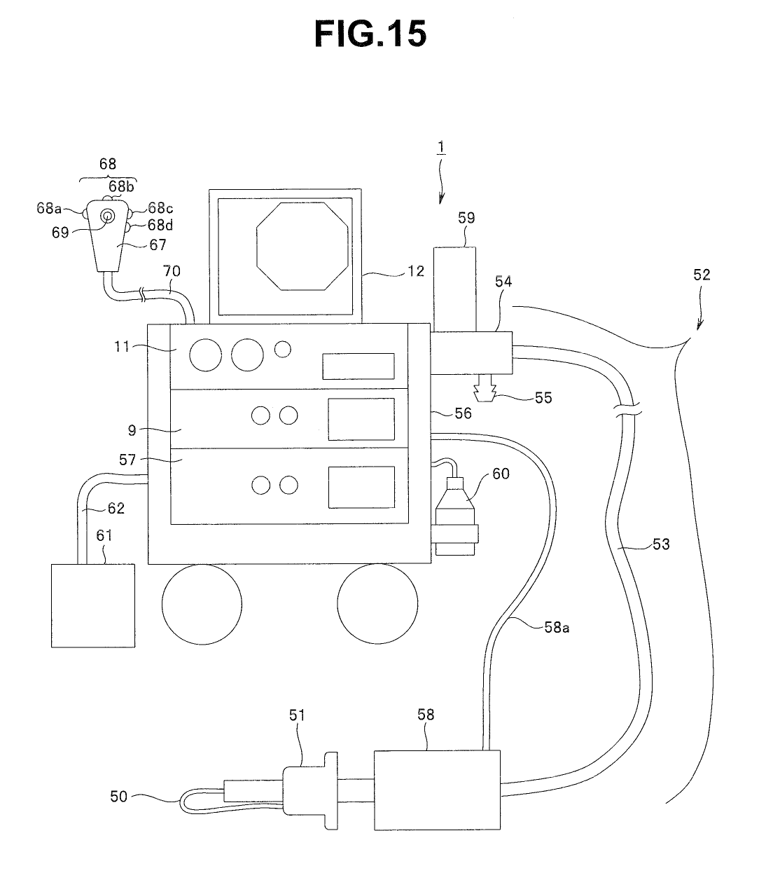

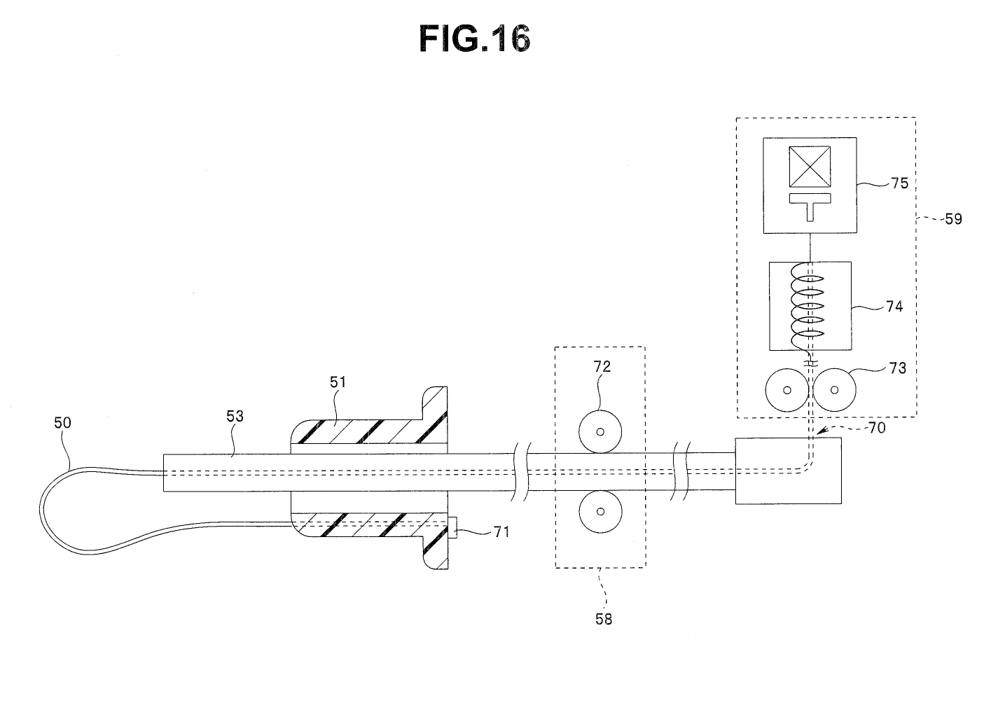

FIG. 15 to FIG. 21 relate to a second embodiment of the present invention, FIG. 15 is a diagram showing an overall configuration of an endoscope apparatus provided with an endoscope insertion aid according to a second embodiment of the present invention and FIG. 16 is a diagram for illustrating the configuration including the guide wire electric drive section and the insertion portion electric drive section making up the main part of the endoscope apparatus in FIG. 15.

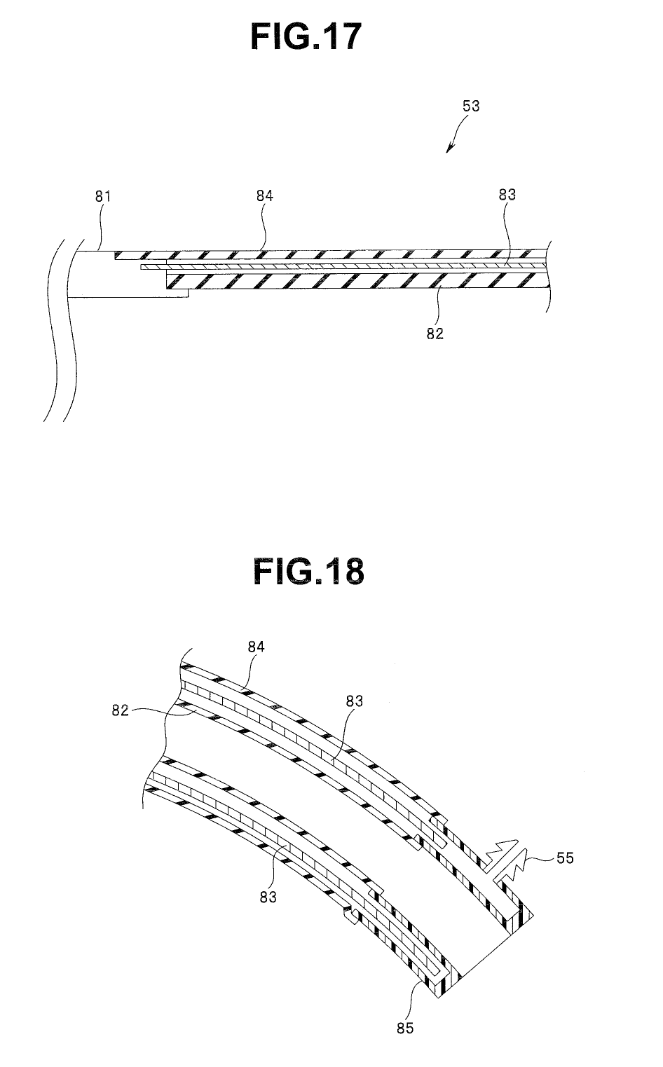

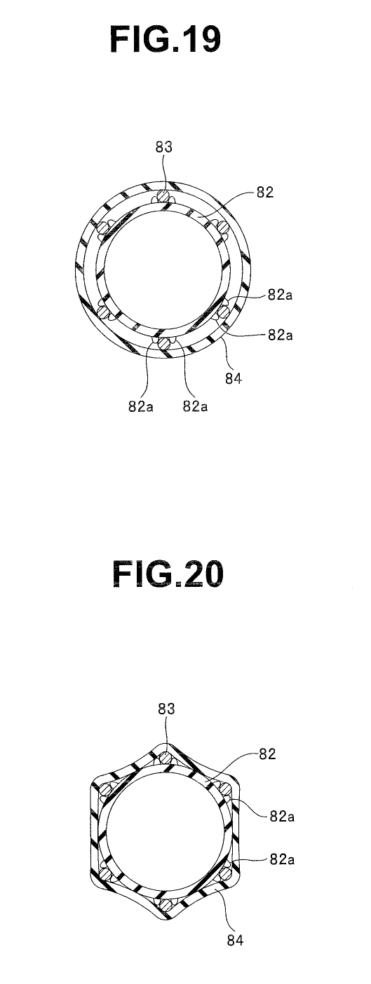

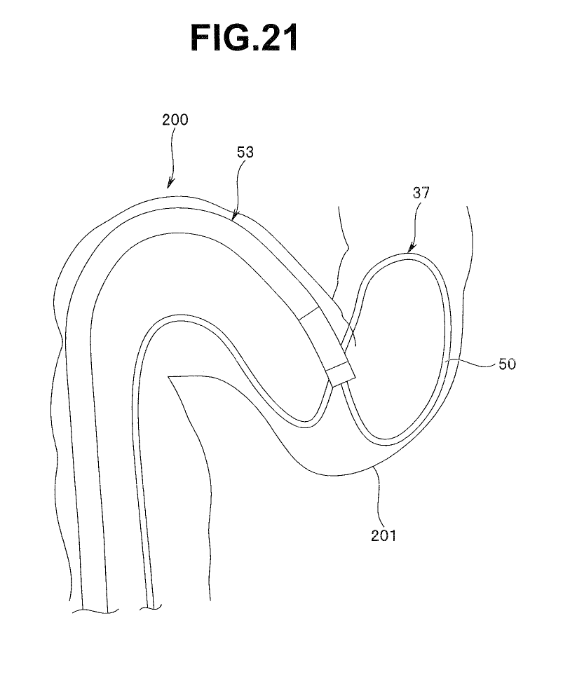

Furthermore, FIG. 17 to FIG. 20 illustrate the configuration of the insertion portion hardness adjusting mechanism provided in the insertion portion in FIG. 14, FIG. 17 is a partially exploded cross-sectional view of the insertion portion on the distal end side, FIG. 18 is a cross-sectional view of the insertion portion on the proximal end side, FIG. 19 is a cross-sectional view in a direction perpendicular to the insertion axial direction when hardness of the insertion portion is in a normal state, FIG. 20 is a cross-sectional view in a direction perpendicular to the insertion axial direction when hardness of the insertion portion is in a high hardness state and FIG. 21 is a diagram for illustrating operations constituting a feature of the second embodiment.

An endoscope apparatus 1 of the present embodiment includes an endoscope insertion aid 51 configured to be substantially the same as that of the first embodiment as shown in FIG. 15, configures an insertion portion 53 passed through the endoscope insertion aid 51 so as to be electrically movable forward or backward and also configures a guide wire portion 50 passed through the insertion portion 53 to be electrically movable forward or backward.

To be more specific, as shown in FIG. 15, the endoscope apparatus 1 is configured by including the endoscope insertion aid 51, guide wire portion 50 configured to be substantially the same as that of the first embodiment, an endoscope 52, an insertion portion electric drive section 58 and a control apparatus 57 that controls a guide wire electric drive section 59.

The endoscope 52 includes the insertion portion electric drive section 58 for electrically moving the insertion portion 53 through which the guide wire portion 50 is passed forward or backward and a guide wire electric drive section 59 for electrically moving the guide wire portion 50 forward or backward.

A connector 54 is connected to a proximal end side of the insertion portion 53 of the endoscope 52. The connector 54 connects the guide wire portion 50 inserted in a treatment instrument insertion channel 33 in the insertion portion 53 and the guide wire electric drive section 59.

Furthermore, the connector 54 is provided with a suction base 55 that communicates with a space in an outer tube 84 (see FIG. 17) of the insertion portion 53, which will be described later, and when the hardness of the insertion portion 53 is made variable, an aspirator 61 is connected to the suction base 55 via a suction tube 62.

The connector 54 is freely attachable to and detachable from a light source device 9 in the same way as in the first embodiment and freely attachable to and detachable from a video processor 11 via a scope cable 10 (not shown).

The insertion portion electric drive section 58 is disposed in the vicinity of the endoscope insertion aid 51 to be attached to the entrance of the intestinal tract, for example, the anus, with the insertion portion 53 inserted therein.

The insertion portion electric drive section 58 and the control apparatus 57 are electrically connected via a cable 58a. Furthermore, the guide wire electric drive section 59 and the control apparatus 57 are electrically connected via a cable (not shown).

The light source device 9, video processor 11 and control apparatus 57 are mounted on a trolley 56. The trolley 56 is provided with a water supply tank 60 that stores water to be supplied during water supply.

Furthermore, the endoscope apparatus 1 is provided with an operation portion 68 for operating the endoscope 2. The operation portion 68 is electrically connected to the video processor 11 and control apparatus 57 via a cable 70.

In addition to an air/water supply button 68c, a suction button 68d and a bending operation portion 69, which are similar to the switches in the first embodiment, the operation portion 68 is also provided with, for example, an insertion portion forward/backward operation button 68a and a guide wire forward/backward operation button 68b. Other operation buttons may also be provided and their respective functions may also be assigned thereto.

The control apparatus 57 controls driving of the light source device 9, video processor 11, aspirator 61, insertion portion electric drive section 58 and guide wire electric drive section 59 or the like based on operation signals from the various operation buttons of the operation portion 68.

Next, the relationship between the insertion portion electric drive section 58, guide wire electric drive section 59 and the insertion portion 53 will be explained using FIG. 16.

As shown in FIG. 16, the insertion portion electric drive section 58 is configured by including a pair of rotation rollers 72 that sandwich, for example, the outer peripheral surface of the insertion portion 53 and a drive motor (not shown), which is a drive source axially supported on at least one of the rotation rollers 72.

Furthermore, the guide wire electric drive section 59 is configured by including a pair of rotation rollers 73 that sandwich, for example, the outer peripheral surface of the guide wire portion 50, a drive motor (not shown), which is a drive source axially supported on at least one of the rotation rollers 73, a guide wire housing section 74 that houses the guide wire portion 50 disposed on the operator's hand side of the rotation roller 73 and a guide wire hardness variable section 75 for making variable the hardness of the guide wire portion 50 disposed on the operator's hand side of the guide wire housing section 74.

The guide wire hardness variable section 75 can make variable the hardness of the guide wire portion 50 by, for example, towing or relaxing the wire 42 of the first embodiment using, for example, a solenoid valve.

The guide wire hardness variable section 75 is electrically connected to the control apparatus 57 via a cable (not shown) to control the current value that flows to the solenoid valve.

Furthermore, when the insertion portion electric drive section 58 and the guide wire electric drive section 59 are simultaneously driven in the present embodiment, the control apparatus 57 controls the guide wire electric drive section 59 so that the forward/backward moving speed of the guide wire portion 50 is twice the forward/backward moving speed of the insertion portion 53 that moves forward or backward electrically driven by the insertion portion electric drive section 58.

The control over the insertion portion electric drive section 58 and the guide wire electric drive section 59 is not limited to such control, and of course, the forward/backward moving speed of the insertion portion 53 and the forward/backward moving speed of the guide wire portion 50 may be changed as required and controlled so as to become optimum speeds as appropriate.

Performing such control allows not only the guide wire portion 50 but also the insertion portion 53 to be smoothly inserted into the depth of the intestinal tract.

In the present embodiment, the insertion portion 53 is configured so that the hardness is freely made variable. The configuration of the insertion portion 53 having such a hardness adjusting mechanism will be explained using FIG. 17 to FIG. 20.

As shown in FIG. 17 and FIG. 18, the insertion portion 53 is configured to be an elongated section with flexibility, and to be more specific, configured by including an inner tube 82, an outer tube 84 coated by forming a space on the outer peripheral side of the inner tube 82 and a wire 83 disposed on the inner peripheral side of the outer tube 84 and in the space between the outer tube 84 and the inner tube 82.

In the configuration of the insertion portion 53 on the distal end side in this case, the respective distal end portions of the inner tube 82, wire 83 and outer tube 84 are fixed to a distal end portion 81 of the insertion portion 53 in order from the inside as shown in FIG. 17.

Furthermore, in the configuration of the insertion portion 53 on the operator's hand side, the respective proximal end portions of the inner tube 82, wire 83 and outer tube 84 are likewise fixed to a rear end portion 85 of the insertion portion 53 in order from the inside as shown in FIG. 18.

The wire 83 is included in the space formed between the inner tube 83 and the outer tube 84.

A suction base 55 that communicates with the space is provided at the rear end portion 85 and the suction base 55 is connected to the aspirator 61 via the suction tube 62.

Operations of the insertion portion hardness adjusting mechanism in such a configuration will be explained using FIG. 19 and FIG. 20.

FIG. 19 shows a cross-sectional view in a direction perpendicular to the insertion axial direction when the hardness of the insertion portion 53 is normal. That is, when the hardness is normal, in other words, when the hardness is not high, the insertion portion 53 maintains predetermined hardness because a space is formed between the inner tube 82 and the outer tube 84, and a predetermined number of wires 83 are arranged in the space.

In FIG. 19, convex portions 82a are provided on the outer peripheral portion of the inner tube 82 along the axial direction of the inner tube 82 and the wires 83 regulate the motion of the inner tube 82 in the circumferential direction.

When the insertion portion 53 is inserted into the intestinal tract, the insertion portion 53 is normally inserted in a state in which such hardness is normal.

Here, when the insertion portion is pushed electrically driven by the insertion portion electric drive section 58 or manually in order to allow the insertion portion to pass through the sigmoid colon portion 200 or bent portion 201 as in the case of the first embodiment, the operator drives the aspirator 61 by operating the operation portion 68 and causes the air in the insertion portion 53 to be suctioned.

When the air in the space is suctioned as shown in FIG. 20, the outer tube 84 of the insertion portion 53 is in a collapsed state, and this causes friction between the wire 83, inner tube 82 and outer tube 83 to increase, resulting in high hardness.

In this case, when the insertion portion 53 is bent, the hardness increases with the wire 83 remaining bent and therefore the bent state, that is, the bent shape is maintained.

Therefore, when the insertion portion 53 is made to pass through the sigmoid colon portion 200 as shown in FIG. 21 or inserted along the guide wire portion 50 that has passed through the bent portion 201 of the sigmoid colon portion 200, the bent shape is maintained with the hardness of the insertion portion 53 set to high hardness, and the guide wire portion 50 can be thereby easily inserted into the depth of the intestinal tract without causing the insertion portion 53 to bend.

The rest of operations are similar to those of the first embodiment.

Therefore, the second embodiment provides the insertion portion electric drive section 58 and the guide wire electric drive section 59, and thereby provides an effect of being able to automatically perform insertion operation in addition to the effects of the first embodiment.

Furthermore, the second embodiment provides the hardness adjusting mechanism in the insertion portion 53 and thereby also has an effect of being easily inserted into the depth of the intestinal tract. The rest of the effects are similar to those of the first embodiment.

The second embodiment has explained the configuration including the insertion portion electric drive section 58, guide wire electric drive section 59 and insertion portion hardness adjusting mechanism, but all these components need not be provided and the present invention can be configured by including any one of the above components.

The present invention is not limited only to the above described embodiments and modification examples, but can be implemented modified in various ways without departing from the gist of the invention.

Having described the preferred embodiments of the invention referring to the accompanying drawings, it should be understood that the present invention is not limited to those precise embodiments and various changes and modifications thereof could be made by one skilled in the art without departing from the spirit or scope of the invention as defined in the appended claims.

What is claimed is:

1. An endoscope insertion aid comprising:
a tube body that is inserted into an entrance of an intestinal tract and held by an intestinal wall;
a passage hole through which an insertion portion of an endoscope can pass, the endoscope having an insertion path through which a guide wire can pass; and
a fixing unit that fixes, with the insertion portion passed through the passage hole, a distal end portion of the guide wire protruding from an opening on a distal end side of the insertion portion to the tube body.

2. An endoscope apparatus comprising:
a guide wire inserted into an intestinal tract;
an insertion portion of an endoscope having an insertion path through which the guide wire is passed; and
an endoscope insertion aid that comprises a passage hole through which the insertion portion can pass, is inserted into an entrance of the intestinal tract and held by an intestinal wall, and disposed, when the insertion portion is inserted into the intestinal tract, closer to a proximal end side than a distal end side of the insertion portion,
wherein the distal end portion of the guide wire protruding from an opening of the insertion path on the distal end side of the insertion portion can be fixed to the endoscope insertion aid.

3. The endoscope apparatus according to claim 2, wherein the endoscope insertion aid comprises a fixing unit that detachably fixes a distal end portion of the guide wire.

4. The endoscope apparatus according to claim 2, further comprising a guide wire drive section for moving a proximal end side of the guide wire forward or backward along a longitudinal direction of the guide wire.

5. The endoscope apparatus according to claim 2, wherein the guide wire is configured to be able to change hardness of a portion within a predetermined range in a longitudinal direction of the guide wire.

6. The endoscope apparatus according to claim 2, wherein the insertion portion is configured such that a shape of the insertion portion can be maintained.

7. The endoscope apparatus according to claim 4, further comprising an insertion portion electric drive section that electrically moves the insertion portion forward or backward in a longitudinal direction.

8. The endoscope apparatus according to claim 7, further comprising a control section that controls the guide wire electric drive section and the insertion portion electric drive section,
wherein the control section controls the guide wire electric drive section so that a forward/backward moving speed of the guide wire is twice a forward/backward moving speed of the insertion portion that moves forward or backward electrically driven by the insertion portion electric drive section.

9. An endoscope apparatus comprising:
a guide wire inserted into an intestinal tract;
an insertion portion of an endoscope having an insertion path through which the guide wire can pass; and
a fixing member that fixes a distal end portion of the guide wire which is inserted into the insertion path to protrude from an opening on a distal end side of the insertion portion and which is bent into a quasi-U shape, disposed outside the insertion portion and closer to a proximal end side than the distal end portion of the insertion portion.

10. An endoscope apparatus insertion method comprising:
a step of causing a guide wire passed through an insertion path in an insertion portion of an endoscope passed through a passage hole of an endoscope insertion aid to protrude from an opening on a distal end side of the insertion portion;
a step of bending a region of a distal end side of the protruding guide wire into a quasi-U shape;
a step of fixing the distal end of the bent guide wire to a fixing unit of the endoscope insertion aid; and
a step of inserting, in a state in which a region protruding from the opening on the distal end side of the insertion portion of the guide wire is bent into a quasi-U shape and the distal end of the guide wire is fixed to the fixing unit, the quasi-U-shaped region of the guide wire and the insertion portion into an intestinal tract.

11. The endoscope apparatus insertion method according to claim 10, wherein a bent portion in the intestinal tract is made to expand by causing the quasi-U-shaped region of the guide wire to move forward in the intestinal tract.

12. The endoscope apparatus insertion method according to claim 11, wherein the quasi-U-shaped region of the guide wire is caused to move forward in the intestinal tract using a guide wire electric drive section.

13. The endoscope apparatus insertion method according to claim 11, wherein the quasi-U-shaped region of the guide wire is caused to move forward in the intestinal tract by making hardness of the guide wire variable.
